SEQUENCE LISTING (34 sequences)

SEQ ID NO: 1 - protein, 5 aa, Artificial Sequence
  Made in a lab
KKKKK

SEQ ID NO: 2 - protein, 7 aa, Artificial Sequence
  Made in a lab
KKKKKKK

SEQ ID NO: 3 - protein, 9 aa, Artificial Sequence
  Made in a lab
KKKKKKKKK

SEQ ID NO: 4 - protein, 15 aa, Artificial Sequence
  Made in a lab
KKKKKKKKKKKKKKK

SEQ ID NO: 5 - protein, 5 aa, Artificial Sequence
  Made in a lab
RVRVR

SEQ ID NO: 6 - protein, 5 aa, Artificial Sequence
  Made in a lab
RRVVR

SEQ ID NO: 7 - protein, 5 aa, Artificial Sequence
  Made in a lab
RRVRR

SEQ ID NO: 8 - protein, 6 aa, Artificial Sequence
  Made in a lab
RRVRVR

SEQ ID NO: 9 - protein, 6 aa, Artificial Sequence
  Made in a lab
RRVVRR

SEQ ID NO: 10 - protein, 7 aa, Artificial Sequence
  Made in a lab
RRRVRRR

SEQ ID NO: 11 - protein, 7 aa, Artificial Sequence
  Made in a lab
RRVRVRR

SEQ ID NO: 12 - protein, 8 aa, Artificial Sequence
  Made in a lab
RRRVVRRR

SEQ ID NO: 13 - protein, 8 aa, Artificial Sequence
  Made in a lab
RRVRRVRR

SEQ ID NO: 14 - protein, 10 aa, Artificial Sequence
  Made in a lab
RRRRVVRRRR

SEQ ID NO: 15 - protein, 10 aa, Artificial Sequence
  Made in a lab
RRVVRRVVRR

SEQ ID NO: 16 - protein, 5 aa, Artificial Sequence
  Made in a lab
RWRWR

SEQ ID NO: 17 - protein, 5 aa, Artificial Sequence
  Made in a lab
RRWWR

SEQ ID NO: 18 - protein, 5 aa, Artificial Sequence
  Made in a lab
RRWRR

SEQ ID NO: 19 - protein, 6 aa, Artificial Sequence
  Made in a lab
RRWRWR

SEQ ID NO: 20 - protein, 6 aa, Artificial Sequence
  Made in a lab
RRWWRR

SEQ ID NO: 21 - protein, 7 aa, Artificial Sequence
  Made in a lab
RRRWRRR

SEQ ID NO: 22 - protein, 7 aa, Artificial Sequence
  Made in a lab
RRWRWRR

SEQ ID NO: 23 - protein, 8 aa, Artificial Sequence
  Made in a lab
RRRWWRRR

SEQ ID NO: 24 - protein, 8 aa, Artificial Sequence
  Made in a lab
RRWRRWRR

SEQ ID NO: 25 - protein, 12 aa, Artificial Sequence
  Made in a lab
GKKEKPEKKVKK

SEQ ID NO: 26 - protein, 16 aa, Artificial Sequence
  Made in a lab
KLTKPKPQAESKKKKK

SEQ ID NO: 27 - protein, 15 aa, Artificial Sequence
  Made in a lab
KKKKKEGKKQEKMLD

SEQ ID NO: 28 - protein, 8 aa, Artificial Sequence
  Made in a lab
KKKDKVKK

SEQ ID NO: 29 - protein, 11 aa, Artificial Sequence
  Made in a lab
KVRQGTLKKAR

SEQ ID NO: 30 - protein, 16 aa, Artificial Sequence
  Made in a lab
PKTKAKAKAKKGKGKD

SEQ ID NO: 31 - protein, 9 aa, Artificial Sequence
  Made in a lab
RRRRRRRRR

SEQ ID NO: 32 - protein, 11 aa, Artificial Sequence
  Made in a lab
RRRRRRRRRRR

SEQ ID NO: 33 - protein, 13 aa, Artificial Sequence
  Made in a lab
RRRRRRRRRRRRR

SEQ ID NO: 34 - protein, 15 aa, Artificial Sequence
  Made in a lab
RRRRRRRRRRRRRRR

(19) United States
(12) Patent Application Publication
(10) Pub. No.: US 2010/0137222 A1
(43) Pub. Date: Jun. 3, 2010

(54) Basic Peptides and Their Use as Combined Antibacterial-Antifungine Agents

(75) Inventor: Deborah O'Neil, Aberdeen (GB)

Correspondence Address: SPECKMAN LAW GROUP PLLC, 1201 THIRD AVENUE, SUITE 330, SEATTLE, WA, 98101, US

(73) Assignee: NOVABIOTICS LIMITED, Aberdeen (GB)

(21) Appl. No.: 12/525,518

(22) PCT Filed: Jan. 28, 2008
(86) PCT No.: PCT/GB08/00282

§ 371 Date: Dec. 15, 2009

Related U.S. Application Data
(60) Provisional application No. 60/899,167, filed on Feb. 2, 2007.

(30) Foreign Application Priority Data
Feb. 2, 2007 (GB) 0702022.5

(51) Int. Cl. A61K 38/10; A61K 38/02; A61K 38/08; A61P 17/00; A61P 17/02; A61P 17/10; A61P 29/00; C07K 2/00

(52) U.S. Cl. 514/14; 514/2; 514/15; 514/16; 514/17; 530/300

(57) ABSTRACT

The invention relates to peptides, and peptide variants thereof, in which substantially all of the amino sequence of said peptide are the same, for use in the treatment of a mixed microbial infection.

### FIELD OF THE INVENTION

This invention relates to peptides and their use in the treatment of diseases or conditions that are associated with a mixed microbial infection.

### BACKGROUND TO THE INVENTION

Many infectious diseases and disorders are multi-factorial and can be caused by, or associated with, a number of different microorganisms.

It has been reported that 25% of burn wounds seen at post-mortem harbour fungi. Other dermal wounds are also often associated with mixed microbial infections. Fungi from burn/topical wound infections may subsequently invade deep tissue and the bloodstream leading to serious complications. Almost all fungal burn infections are mixed with bacterial infections; in rare cases Candida spp infections alone have been reported. The predominant fungi recovered from burns are Aspergillus spp and Candida spp. The most common bacteria infecting wounds are Staphylococcus aureus, Pseudomonas aeruginosa, Escherichia coli, Proteus mirabilis, Enterococcus spp, Enterobacter spp, Acinetobacter spp, Klebsiella spp and β-haemolytic Group A Streptococci.

Cystic fibrosis (CF) is most commonly associated with bacterial infection, especially Ps aeruginosa and Burkholderia cepacia complex. In addition to bacterial infection, co-existing respiratory fungal infections are relatively common in CF patients, such as A fumigatus, C albicans, Scedosporium apiospermum and Pneumocystis jirovecii.

Paronychia refers to inflammation of the nail fold. Inflammation in chronic paronychia is caused by several different micro-organisms. Often a mixture of yeasts and bacteria are present, particularly Candida spp and Gram negative bacilli. The inflammation results in debris which builds up, encouraging more infection. It mainly occurs in people who have constantly wet hands, such as dairy farmers, fishermen, bar tenders and housewives. It is more likely to occur, and more difficult to clear up, in those with poor circulation, especially during the winter months. It can also be a complication of eczema.

Microbial keratitis is an infection of the cornea, most commonly associated with contact-lens use and in under-developed nations. Most microbial keratitis is bacterial, but up to 8% of cases can be caused by mixed bacterial and fungal infections.

Infective mastitis is an inflammation of the breast caused by an infectious agent. Infective mastitis is normally caused by S. aureus, and is most common in breast-feeding mothers (and cows). Mastitis can also be caused by yeasts (especially Candida spp), and in some cases mixed bacterial/yeast infections.

There is, therefore, a need for improved therapies that are effective in treating mixed infectious diseases and conditions such as those described herein.

Acne is a very common inflammatory disease of the skin caused by the hyperproliferation of skin cells (keratinocytes) combined with excessive secretion of oil from the sebaceous glands (Gollnick, 2003). The sebum becomes stagnant beneath the blocked hair follicles, which provides ideal conditions for the uncontrolled multiplication of commensal skin microorganisms represented by three groups: the Gram-positive cocci, including Staphylococcus aureus; the anaerobic diphteroids such as Propionibacterium acnes; and the lipophilic yeasts including Malassezia furfur (Bukhart et al., 1999). Amongst those microorganisms, only P. acnes seems to play a role in the pathogenesis of acne (Bukhart et al., 1999). Furthermore, P. acnes has been associated with inflammatory reactions and was shown to up-regulate the pro-inflammatory pathways in the human skin cells (Basal et al., 2004; Nagy et al., 2005; Nagy et al., 2006; Trivedi et al., 2006). Nevertheless, mixed microbial infections are commonly found in acne lesions.

Mild-to-moderate cases of acne are commonly treated with topical therapies, whereas systemic medications and combination of topical and systemic therapies are usually applied to more severe cases. The most commonly therapies used so far against acne have been based on antibiotics and chemical, (e.g. anti-inflammatory) agents or a combination of the two. There is increasing evidence, however, that antibiotic resistance in P. acnes clinical isolates is rising, which jeopardizes the use of antibiotics for the treatment of acne in the future (Coates et al., 2002; Ochsendorf, 2006). Moreover, antibiotics will not be effective against fungal organisms associated with acne. Furthermore, certain non-antibiotic acne therapies are associated with some very serious (e.g. psychological) side-effects. There is a very acute clinical and economic need therefore, for the development of alternative anti-acne therapies, with a significant emphasis on safety, as well as broad-spectrum antimicrobial effectiveness.

### STATEMENTS OF THE INVENTION

The present invention is based in part on the finding that homopolymers of arginine or lysine are both bactericidal and fungicidal and as such are highly effective in the treatment of a mixed fungal and bacterial associated disease or condition.

According to a first aspect the invention provides a peptide for use in the combined treatment of a bacterial and fungal infection wherein substantially all of the amino acids in the amino sequence of said peptide are the same.

In a preferred aspect of the invention the amino acids of said sequence are basic amino acids.

In a preferred peptide the basic amino acids are selected from lysine, arginine and histidine, in particular lysine and arginine. Preferably still the basic amino acid is arginine.

As used herein “substantially” is a relative modifier intended to indicate permissible variation from the characteristic so modified. Specifically, by “substantially all of the amino acids in said amino acid sequence are the same” it is meant that either all, or a high proportion of, the amino acids in the sequence are identical. By “high proportion” it is contemplated that 1 or 2 substitutions may be made in the sequence.

In a preferred aspect the invention provides a peptide, or peptide variant thereof, comprising an amino acid sequence according to the formula (I)

(X) (I)

wherein X is a basic amino acid for use as a medicament.

In a peptide of the invention X may be arginine.

In a peptide of the invention X may be lysine.

In a peptide of the invention X may be histidine.

In a preferred aspect of the invention the peptide comprises a sequence of 5 to 15 basic amino acids.

In a preferred aspect the peptide of the invention comprises a sequence of 9 to 15, for example 10 to 15 or 10 to 13, basic amino acids wherein substantially all of the amino acids in said sequence of amino acids are the same. Preferably still the peptide of the invention comprises a sequence of 9 to 13, for example 11 to 13, basic amino acids wherein substantially all of the amino acids in said sequence are the same.

In a preferred aspect the invention provides a peptide, or peptide variant thereof, comprising an amino acid sequence according to the formula (II)

(X)ₙ (II)

wherein X is the amino acid arginine or lysine and n is an integer between 5 and 15, for use as a medicament.

In a preferred peptide of the invention X is arginine.

In a preferred peptide of the invention X is lysine.

In a peptide of the invention n may be between 9 and 15 e.g. 9, 10, 11, 12, 13, 14 or 15. In a preferred peptide of the invention n is between 9 and 14, for example between 10 and 14. Preferably n is between 11 and 14 for example.

In an alternative preferred peptide of the invention n is an integer between 9 and 12, for example 9, 10 or 11. Preferably still n is 11.

In a peptide of formula (I), X may be a D- or L-amino acid.

In a preferred aspect the invention provides a linear peptide consisting of amino acids according to formula (I).

The invention also includes known isomers (structural, stereo-, conformational & configurational) and structural analogues of the above amino acids, including peptidomimetics, and those modified either naturally (e.g. post-translational modification) or chemically, including, but not exclusively, phosphorylation, glycosylation, sulfonylation and/or hydroxylation.

In addition, the amino acid sequence of the peptide can be modified so as to result in a peptide variant that includes the substitution of at least one amino acid residue in the peptide for another amino acid residue, for example a basic or non-basic residue, including substitutions that utilise the D rather than L form, wherein the variant retains some (typically at least 10%) or all of the biological activity of the corresponding non-variant peptide. Thus the invention provides a peptide variant in which one or more lysine or arginine residues of formula (I) is substituted by one or more different (e.g. histidine) residues.

The term “peptide” as used herein means, in general terms, a plurality of amino acid residues joined together by peptide bonds. It is used interchangeably and means the same as polypeptide, oligopeptide and protein.

The peptides of the invention generally are synthetic peptides. The peptides may be isolated, purified peptides or variants thereof, which can be synthesised in vitro, for example, by a solid phase peptide synthetic method, by enzyme catalysed peptide synthesis, or with the aid of recombinant DNA technology.

The peptides of the invention can exist in different forms, such as free acids, free bases, esters and other prodrugs, salts and tautomers, for example, and the invention includes all variant forms of the peptides. Thus, the invention encompasses the salt or pro-drug of a peptide.

The peptide of the invention may be administered in the form of a pharmaceutically acceptable salt. The invention thus includes pharmaceutically-acceptable salts of the peptide of the invention wherein the parent compound is modified by making acid or base salts thereof for example the conventional non-toxic salts or the quaternary ammonium salts which are formed, e.g., from inorganic or organic acids or bases. Examples of such acid addition salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, oxalate, palmoate, pectinate, persulfate, 3-phenylpropionate, picrate, pivalate, propionate, succinate, tartrate, thiocyanate, tosylate, and undecanoate. Base salts include ammonium salts, alkali metal salts such as sodium and potassium salts, alkaline earth metal salts such as calcium and magnesium salts, salts with organic bases such as dicyclohexylamine salts, N-methyl-D-glutamine, and salts with amino acids such as arginine, lysine, and so forth. Also, the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl bromides and others.

Salts of carboxyl groups of a peptide or peptide variant of the invention may be prepared in the usual manner by contacting the peptide with one or more equivalents of a desired base such as, for example, a metallic hydroxide base, e.g. sodium hydroxide; a metal carbonate or bicarbonate such as, for example, sodium carbonate or bicarbonate; or an amine base such as, for example, triethylamine, triethanolamine and the like.

Administration and Pharmaceutical Formulations

A further aspect of the invention provides a pharmaceutical composition comprising a pharmaceutically effective amount of one or more peptides of the invention.

The composition also includes a pharmaceutically acceptable carrier, excipient or diluent. The phrase “pharmaceutically acceptable” is employed herein to refer to those compounds, materials, compositions, and/or dosage forms which are, within the scope of sound medical judgment, suitable for use in contact with the tissues of human beings or, as the case may be, an animal without excessive toxicity, irritation, allergic response, or other problem or complication, commensurate with a reasonable benefit/risk ratio.

To achieve the desired effect(s), the peptide, a variant thereof or a combination thereof, may be administered as single or divided dosages, for example, of at least about 0.01 mg/kg to about 500 to 750 mg/kg, of at least about 0.01 mg/kg to about 300 to 500 mg/kg, at least about 0.1 mg/kg to about 100 to 300 mg/kg or at least about 1 mg/kg to about 50 to 100 mg/kg of body weight or at least about 1 mg/kg to about 20 mg/kg of body weight, although other dosages may provide beneficial results.

To prepare the composition, peptides are synthesized or otherwise obtained, purified as necessary or desired, and then lyophilized and stabilized. The peptide can then be adjusted to the appropriate concentration and optionally combined with other agents.

Thus, one or more suitable unit dosage forms comprising the therapeutic peptides of the invention can be administered by a variety of routes including oral, topical, parenteral (including subcutaneous, intravenous, intramuscular and intraperitoneal), vaginal, rectal, dermal, transdermal, intrathoracic, intrapulmonary and intranasal (respiratory) routes. The therapeutic peptides may also be formulated in a lipid formulation or for sustained release (for example, using microencapsulation, see WO 94/07529, and U.S. Pat. No. 4,962,091). The formulations may, where appropriate, be conveniently presented in discrete unit dosage forms and may be prepared by any of the methods well-known to the pharmaceutical arts. Such methods may include the step of mixing the therapeutic agent with liquid carriers, solid matrices, semi-solid carriers, finely divided solid carriers or combinations thereof, and then, if necessary, introducing or shaping the product into the desired delivery system.

When the therapeutic peptides of the invention are prepared for oral administration, they are generally combined with a pharmaceutically acceptable carrier, diluent or excipient to form a pharmaceutical formulation, or unit dosage form. For oral administration, the peptides may be present as a powder, a granular formation, a solution, a suspension, an emulsion.

Pharmaceutical formulations containing the therapeutic peptides of the invention can be formulated with common excipients, diluents, or carriers, and formed into tablets, capsules, solutions, suspensions, powders, aerosols and the like.

The therapeutic peptides of the invention can also be formulated as elixirs or solutions for convenient oral administration or as solutions appropriate for parenteral administration, for instance by intramuscular, subcutaneous, intraperitoneal or intravenous routes. The pharmaceutical formulations of the therapeutic peptides of the invention can also take the form of an aqueous or anhydrous solution or dispersion, or alternatively the form of an emulsion or suspension or salve.

The therapeutic peptides may be formulated for parenteral administration (e.g. by injection, for example, bolus injection or continuous infusion) and may be presented in unit dose form in ampules, pre-filled syringes, small volume infusion containers or in multi-dose containers.

These formulations can contain pharmaceutically acceptable carriers, vehicles and adjuvants that are well-known in the art. It is possible, for example, to prepare solutions using one or more organic solvent(s) that is/are acceptable from the physiological standpoint, chosen, in addition to water, from solvents such as acetone, acetic acid, ethanol, isopropyl alcohol, dimethyl sulphoxide, glycol ethers such as the products sold under the name “Dowanol”, polyglycols and polyethylene glycols, C₁-C₄ alkyl esters of short-chain acids, ethyl or isopropyl lactate, fatty acid triglycerides such as the products marketed under the name “Miglyol”, isopropyl mytrisate, animal, mineral and vegetable oils and polysiloxanes.

A preferred route of administration is topical. For topical administration, the peptides may be formulated as is known in the art for direct application to a target area, for example nails and skin. Forms chiefly conditioned for topical application take the form, for example, of laquers, creams, milks, gels, powders, dispersion or microemulsions, lotions thickened to a greater or lesser extent, impregnated pads, ointments or sticks, aerosol formulations (e.g. sprays or foams), soaps, detergents, lotions or cakes of soap. Other conventional forms for this purpose include wound dressings, coated bandages or other polymer coverings, ointments, creams, lotions, pastes, jellies, sprays, and aerosols. Thus, the therapeutic peptides of the invention can be delivered via patches or bandages for dermal administration.

It may be possible to administer a peptide of the invention transdermally via, for example, some form of transdermal delivery device. Such devices are advantageous, particularly for the administration of antibiotic compounds, as they may allow a prolonged period of treatment relative to for example, an oral or intravenous medicament. Examples of transdermal delivery devices may include, for example, a patch, dressing, bandage or plaster adapted to release the peptide through the skin of a patient. A person of skill in the art would be familiar with the materials and techniques which may be used to transdermally deliver a compound or substance and exemplary transdermal delivery devices are provided by GB2185187, U.S. Pat. No. 3,249,109, U.S. Pat. No. 3,598,122, U.S. Pat.4,144,317, U.S. Pat. No. 4,262,003 and U.S. Pat. No. 4,307,717. By way of example, a peptide of the invention may be combined with some form of matrix or substrate, such as a non-aqueous polymeric carrier, to render it suitable for use in a transdermal delivery system. The peptide/matrix or substrate mixture may be further strengthened by the use of a woven or knit, non-woven, relatively open mesh fabric, to produce a patch, bandage, plaster or the like which may be temporarily attached to a particular region of a patient's body. In this way, while in contact with a patient's skin, the transdermal delivery device releases the compound or substance directly to the site of infection or through the skin as required.

The peptides of the invention may also be used as sterilising or cleaning aids for use, for example, on surfaces to reduce and/or eliminate contamination by bacteria. For example, peptides of the present invention may be added to or diluted in an appropriate excipient or solution prior to use as a sterilising or cleaning agent. Exemplary excipients are described above. Such sterilising or cleaning solutions may be used to decontaminate, for example, furniture, floors, equipment including for example specialised hospital equipment and/or surgical equipment. In a further embodiment, the peptides of the invention may be used to eliminate and/or reduce bacterial contamination on parts of the body, particularly for example, the hands. The peptide may be diluted as an aqueous or non-aqueous solution (dissolved in aqueous, non aqueous or organic solvent) and which may be applied to a body part, for example the hands.

The peptides of the invention can also be administered to the respiratory tract. Thus, the present invention also provides aerosol pharmaceutical formulations and dosage forms for use in the methods of the invention. In general, such dosage forms comprise an amount of at least one of the agents of the invention effective to treat or prevent the clinical symptoms of a specific infection, indication or disease. Any statistically significant attenuation of one or more symptoms of an infection, indication or disease that has been treated pursuant to the method of the present invention is considered to be a treatment of such infection, indication or disease within the scope of the invention.

The peptides of the invention may be provided as a combination therapy together with one or more known antimicrobial agents. Typically the peptides of the invention are provided as a monotherapy for the treatment of an infection.

Use

The peptides of the invention may be useful in the treatment or prevention of a disease or condition that is contributed to or caused by an infection by at least two different identifiable microorganisms from different kingdoms or genera, otherwise termed a “mixed microbial infection”. The microorganisms may be bacteria, fungi (including yeast), viruses or parasites. Preferably the microorganisms are bacteria and fungi. The disease causing microorganisms may be obligate or opportunistic pathogens.

Preferably the peptides of the invention are useful in the simultaneous treatment or prevention of an infection contributed to or caused by a bacterium and a fungus.

Thus a further aspect of the invention provides the use of a peptide according to the invention, or a pharmaceutically acceptable salt thereof, in the manufacture of a medicament for the treatment or alleviation of an infection contributed to or caused by a bacterium and a fungus.

The bacterium may be either a Gram positive or Gram negative bacterium. The bacterium may be selected from, but not limited to, Propionibacteria spp. (e.g. Propionibacterium acnes), Bacillus spp, Staphylococcus spp (e.g. Staphylococcus aureus), Pseudomonas spp, (e.g. Pseudomonas aeruginosa), Escherichia coli, Proteus mirabilis, Enterococcus spp, Enterobacter spp, Acinetobacter spp, Klebsiella spp and Streptococcus spp. (e.g. Group A β-haemolytic streptococci).

The bacterium may be Propionibacterium acnes.

The fungus may be any fungus for example a fungus selected from the group consisting of, but not limited to, Absidia spp, (e.g. Absidia corymbifera), Aspergillus spp, (e.g. Aspergillus candidus, Aspergillus niger, Aspergillus tamarii, Aspergillus flavus, Aspergillus fumigatus, Aspergillus sydowii, Aspergillus terreus, Aspergillus ustus, Aspergillus versicolor, Aspergillus clavatus, Aspergillus glaucus group, Aspergillus nidulans, Aspergillus oryzae,), Cryptococcus spp (e.g. Crytococcus neoformans var. neoformans, Crytococcus neoformans var. gatii, Crytococcus neoformans var. grubii), Malassezia spp (e.g. Malassezia furfur, Malassezia pachydermatis, Malassezia globosa, Malassezia obtuse, Malassezia restricta, Malassezia slooffiae, Malassezia sympodialis), Candida spp (e.g. Candida albicans, Candida tropicalis, Candida glabrata, Candida parapsilosis, Candida krusei, Candida lusitaniae, Candida kefyr, Candida sake, Candida guilliermondii, Candida dubliniensis, Candida ciferii, Candida famata, Candida lambica, Candida lipolytica, Candida norvegensis, Candida rugosa, Candida viswanathii, Candida zeylanoides), Rhizomucor spp, (e.g. Rhizomucor pusillus, Rhizomucor miehei, Rhizomucor variabilis), Saccharomyces spp, (e.g. Saccharomyces cerevisiae, Saccharomyces boullardii), Hansenula spp, Fusarium spp (e.g. Fusarium oxysporum, Fusarium solani, Fusarium chlamydosporum, Fusarium moniliforme, Fusarium proliferatum), Mucor spp (e.g. Mucor amphibiorum, Mucor circinelloides, Mucor hiemalis, Mucor indicus, Mucor racemosus, Mucor ramosissimus), Trichosporon spp (e.g. Trichosporon beigelii, Trichosporon cutaneum, Trichosporon asteroids, Trichosporon ovoides, Trichosporon inikin, Trichosporon asahii, Trichosporon mucoides), Rhodotorula spp, (e.g. Rhodotorula glutinis, Rhodotorula minuta, Rhodotorula mucilaginosa), Pichia spp, (e.g. Pichia anomola, Pichia guilliermondii, Pichia norvegensis, Pichia ohmerii), Rhizopus spp., (e.g. Rhizopus arrhizus, Rhizopus microsporus), Penicillium spp (e.g. Penicillium marneffei, Penicillium verrucosum) and Blastoschizomyces spp (e.g. Blastoschizomyces capitatus).

The fungus may be Malassezia spp, for example, Malassezia furfur.

The invention further provides the use of a peptide of the invention, or a pharmaceutically acceptable salt thereof, in the manufacture of a medicament for the treatment or alleviation of a disease or condition contributed to or caused by an infection of a bacterium and a fungus. For example, the disease or condition may be selected from the group consisting of acne (acne vulgaris), paronychia, microbial keratitis, mastitis, topical wounds, burns (including sunburn and thermal burns), minor inflammatory conditions caused by a bacterial and/or fungal infection and cystic fibrosis.

The disease or condition to be treated may be of the nail, skin, dermis, breast, cornea or lungs in particular the skin, dermis or the lung.

In a use according to the invention the disease or condition may be acne.

In a use according to the invention the disease or condition may be paronychia.

In a use according to the invention the disease or condition may be a wound.

In a use according to the invention the disease or condition may be an ulcer.

In a use according to the invention the disease or condition may be a burn.

In a use according to the invention the disease or condition may be cystic fibrosis.

In one embodiment the invention provides the use of a peptide of formula (I) wherein n is an integer between 11 and 15, in particular between 11 and 13, or a pharmaceutically acceptable salt thereof, in the manufacture of a medicament for the treatment or alleviation of a disease or condition contributed to or caused by an infection of a bacterium and a fungus.

The peptides of the invention are effective in the simultaneous treatment of a disease or condition that is contributed to or caused by an infection by both a bacterium and a fungus. Thus, the invention provides a method for the treatment, prevention or delay of progression of a disease or condition that is contributed to or caused by a mixed microbial infection which comprises administering to a patient a therapeutically effective amount of a peptide according to the invention, or a pharmaceutically acceptable salt thereof. Preferably the patient is a mammal, in particular human.

The route of administration of the peptide, or pharmaceutically acceptable salt thereof, may be topical, oral, aerosol, intradermal, intramuscular or intravenous administration.

In a preferred method of the invention the peptide, or pharmaceutically acceptable salt thereof, is administered topically. For example, topical administration is preferable where the disease or condition is of the skin or dermis for example acne, wounds or burns.

Where the route of administration is topical, the peptide or peptide, or pharmaceutically acceptable salt thereof, may be administered using a non-sticking gauze, a bandage, a swab, cloth wipe, a patch, a mask, a protectant, a cleanser, an antiseptic, a solution, a cream, a lotion, an ointment, a gel or an emulsion, a liquid, a paste, a soap or a powder.

In a method of the invention, the peptide, or pharmaceutically acceptable salt thereof, is intended as a formulation intended for inhalation or parenteral administration.

Thus in one embodiment the invention provides a method for the treatment, prevention or delay of progression of a mixed microbial infection which comprises administering to a patient a therapeutically effective amount of an aerosol formulation comprising a peptide according to the invention, or a pharmaceutically acceptable salt thereof. The invention further provides an aerosol formulation, including an inhaler comprising said aerosol formulation, comprising a peptide according to the invention, or a pharmaceutically acceptable salt thereof. For example, inhalation as a mode of administration is preferable where the disease or condition is of the lung for example cystic fibrosis.

The diagnosis of specific diseases and conditions treatable according to the invention can be readily determined by the skilled person by the isolation of the causative microorganism from blood, tissue, urine etc.

The extent of protection includes counterfeit or fraudulent products which contain or purport to contain a compound of the invention irrespective of whether they do in fact contain such a compound and irrespective of whether any such compound is contained in a therapeutically effective amount.

Features, integers, characteristics, compounds, chemical moieties or groups described in conjunction with a particular aspect, embodiment or example of the invention are to be understood to be applicable to any other aspect, embodiment or example described herein unless incompatible therewith.

The following Example illustrates the invention.

### EXAMPLE

Materials and Methods

Peptide Synthesis

All peptides were produced either by solid-phase synthesis under contract by a peptide supplier, NeoMPS SA (Strasbourg, France), or purchased from Sigma-Aldrich Chemical Company Ltd. (Poole, UK).

Sequence of Cationic Peptides

The sequence of the peptides analysed is shown in Tables 1 and 2. Ac represents an Acetylated modification to the C-terminus of the oligopeptide and NH₂ represents an amidation of the N-terminus of the oligopeptide.

Broth Dilution Antibacterial Susceptibility Testing

The sensitivity of relevant bacterial strains to peptides was determined using Clinical Laboratory Standard Institute (CLSI; formerly NCCLS) Approved Standards. Bacterial susceptibility was tested using “Methods for Dilution Antimicrobial Susceptibility Tests for Bacteria That Grow Anaerobically; Approved Standard—Seventh Edition M7-A7”

Broth Dilution Antifungal Susceptibility Testing

The sensitivity of relevant fungal strains to peptides was determined using Clinical Laboratory Standard Institute (CLSI; formerly NCCLS) Approved Standards. Yeast susceptibility was tested using “Reference Method for Broth Dilution Antifungal Susceptibility Testing of Yeasts; Approved Standard—Second Edition M27-A”.

Results

TABLE 1
Antimicrobial Activity of peptides against Acne-associated Microbes.

| | | | Antimicrobial Activity (MIC; mg/ml) | | |
|---|---|---|---|---|---|
| | | Molecular | | P. acnes | |
| | | Weight | P. acnes | clinical isolates | M. furfur |
| Novapeptide | Sequence | (kDa) | DSM1897 | (n) | DSM6170 |
| NP106 | (I-K)n, n = 7-27, HBr salt | 1-4 | 0.125 | 0.0625-0.25 (4) | 0.5 |
| NP101 | (I-K)n, n = 100-200, HCl salt | 15-30 | 0.1 | 0.005-0.01 (10) | |
| NP342 | KKK | | >10* | | 1* |
| NP343 | KKKKK | | >10* | | |
| NP344 | KKKKKKK | | >10* | | 1* |
| NP345 | KKKKKKKKK | | 2.5* | | |
| NP348 | KKKKKKKKKKKKKKK | | 0.0625* | | 0.25* |

(n) shows the number of P. acnes clinical isolates tested
(*value in mM)

TABLE 2
Antimicrobial Activity of Peptides against Acne-associated Microbes.

| | | | Antimicrobial Activity (MIC; mM) | | | |
|---|---|---|---|---|---|---|
| | | Molecular | | P. acnes | | |
| | | Weight | P. acnes | clinical isolates | | M. furfur |
| Novapeptide | Sequence | (Da) | DSM1897 | (n) | | DSM6170 |
| NP301 | RVRVR | 684.9 | >2 | | | |
| NP302 | RRVVR | 684.9 | >2 | | | |
| NP303 | RRVRR | 741.9 | >2 | | | |
| NP304 | RRVRVR | 841 | >2 | | | |
| NP305 | RRVVRR | 841 | >2 | | | |
| NP306 | RRRVRRR | 1054.3 | 2 | | | |
| NP307 | RRVRVRR | 997.2 | >2 | | | |
| NP308 | RRRVVRRR | 1153.4 | >2 | | | |
| NP309 | RRVRRVRR | 1153.4 | 1 | >2 | (4) | |
| NP310 | RRRRVVRRRR | 1465.8 | 0.5 | >2 | (4) | 0.5 |
| NP311 | RRVVRRVVRR | 1351.7 | 2 | >2 | (4) | |
| NP320 | RWRWR | 859 | 1 | | | |
| NP321 | RRWWR | 859 | 1 | | | |
| NP322 | RRWRR | 829 | 2 | | | |
| NP323 | RRWRWR | 1015.2 | 0.5 | | | |
| NP324 | RRWWRR | 1015.2 | 0.25 | | | |
| NP325 | RRRWRRR | 1141.4 | 2 | | | |
| NP326 | RRWRWRR | 1171.4 | 0.25 | | | |
| NP327 | RRRWWRRR | 1327.6 | 0.125 | 1-2 | (4) | |
| NP328 | RRWRRWRR | 1327.6 | 0.5 | | | |
| NP331 | GKKEKPEKKVKK | 1426.8 | >2 | | | |
| NP332 | KLTKPKPQAESKKKKK | 1867.3 | >2 | | | |
| NP333 | KKKKKEGKKQEKMLD | 1846.3 | >2 | | | |
| NP334 | KKKDKVKK | 1001.3 | >2 | | | |
| NP335 | KVRQGTLKKAR | 1284.6 | >2 | | | |
| NP336 | PKTKAKAKAKKGKGKD | 1684.1 | >2 | | | |
| NP316 | RRRRRRRRR | 1423.7 | 0.0313 | 1->2 | (4) | 0.125 |
| NP317 | RRRRRRRRRRR | 1736.1 | <0.0156 | | | <0.0156 |
| NP337 | RRRRRRRRRRRRR | 2048.4 | 0.0025 | 0.0156-1 | (10) | 0.03125 |
| NP319 | RRRRRRRRRRRRRRR | 2360.8 | <0.0156 | <0.0156 | (4) | 0.25 |
| NP338 | Ac-RRRRRRRRRRRRR-NH2 | 2088 | 0.005 | 0.001-0.02 | (10) | <0.0156 |
| NP339 | dRdRdRdRdRdRdRdRdRdRdRdRdR | 2047.7 | 0.02 | 0.001-0.005 | (3) | 0.008 |
| NP340 | Ac-dRdRdRdRdRdRdRdRdR | 2088.9 | 0.02 | 0.0025-0.005 | (3) | 0.008 |
| | dRdRdRdR-NH2 | | | | | |

(n) shows the number of P. acnes clinical isolates tested.

TABLE 3

| Identifier | Amino acid sequence | MIC (mM) |
|---|---|---|
| NP310 | RRRRVVRRRR | 0.5 |
| NP316 | RRRRRRRRR | 0.125 |
| NP317 | RRRRRRRRRRR | <0.0156 |
| NP338 | Ac-RRRRRRRRRRRRR-NH2 | <0.0156 |
| NP121 | (R)n, n = 28-86, HCl salt (5-15 kDa) | 0.25* |
| NP122 | (R)n, n = 86-402, HCl salt (15-70 kDa) | 0.25* |
| NP131 | (H)n, n = 32-161, HCl salt (5-25 kDa) | 0.0625* |
| NP132 | (H)n, n = >32, HCl salt (>5 kDa) | 0.0625* |

Minimum Inhibitory Concentrations (MIC) of the peptides against Malassezia furfur DSM6170 (all peptides are L isomers, unless indicated otherwise (D).
*value in mg/ml).

1-2. (canceled)

3. The method of claim 24 wherein the basic amino acids are selected from lysine, arginine and histidine.

4. The method of claim 24 wherein the basic amino acids are lysine and arginine

5. (canceled)

6. The method of claim 24 wherein the peptide comprises a sequence of 5 to 15 basic amino acids.

7. The method of claim 6 wherein X is arginine.

8. The method of claim 6 wherein X is lysine.

9. The method of claim 6 wherein X is histidine.

10-12. (canceled)

13. The method of claim 24 wherein the disease or condition is selected from the group consisting of: acne, paronychia, microbial keratitis, mastitis, topical wounds, burns, inflammatory conditions caused by a bacterial and/or fungal infection and cystic fibrosis.

14. The method of claim 13 wherein the disease or condition is acne.

15. The method of claim 13 wherein the disease or condition is paronychia.

16. The method of claim 13 wherein the disease or condition is a wound.

17. The method of claim 16 wherein the wound is a burn.

18. The method of claim 16 wherein the wound is an ulcer.

19. The method of claim 16 wherein the disease or condition may be cystic fibrosis.

20. (canceled)

21. A method as claimed in claim 24 wherein the peptide, or pharmaceutically acceptable salt thereof, is administered topically.

22. A method as claimed in claim 24 wherein the peptide, or pharmaceutically acceptable salt thereof, is administered by inhalation.

23. An aerosol formulation comprising a peptide, or a pharmaceutically acceptable salt thereof, wherein substantially all the amino acids in the peptide are the same and are basic amino acids.

24. A method for the treatment, prevention or alleviation of a disease or condition contributed to or caused by an infection of a bacterium and a fungus in a subject, comprising administering to the subject a peptide, or a pharmaceutically acceptable salt thereof, wherein substantially all the amino acids in the peptide are the same and are basic amino acids.
